(19) United States
(12) Patent Application Publication
(10) Pub. No.: US 2009/0047744 A1
(43) Pub. Date: Feb. 19, 2009

(54) Method for Improving the Characterisation of a Polynucleotide Sequence

(75) Inventor: Preben Lexow, Oslo (NO)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Lingvitae AS, Oslo (NO)

(21) Appl. No.: 11/817,177

(22) PCT Filed: Mar. 1, 2006
(86) PCT No.: PCT/GB06/00719

§ 371 Date: May 28, 2008

(30) Foreign Application Priority Data
Mar. 1, 2005 (GB) 0504182.7

(51) Int. Cl. G01N 33/00

(52) U.S. Cl. 436/94

(57) ABSTRACT

A method of identifying at least one characteristic of a target molecule comprises the steps of: (i) converting the at least one characteristic into a signal polynucleotide; and (ii) identifying the signal polynucleotide sequence, thereby identifying the at least one characteristic of the target molecule wherein each signal polynucleotide comprises at least one control sequence that defines a characteristic of the signal polynucleotide, and wherein identification of the control sequence confirms whether the signal polynucleotide sequence has been identified correctly, and, optionally, if the identification is not correct, provides the necessary information to determine what the correct signal polynucleotide sequence should be.

### FIELD OF THE INVENTION

This invention relates to a method for improving the accuracy in characterising a polynucleotide sequence.

### BACKGROUND TO THE INVENTION

Advances in the study of molecules have been led, in part, by improvement in technologies used to characterise the molecules or their biological reactions. In particular, the study of the nucleic acids DNA and RNA has benefited from developing technologies used for sequence analysis and the study of hybridisation events.

The principal method in general use for large-scale DNA sequencing is the chain termination method. This method was first developed by Sanger and Coulson (Sanger et al., Proc. Natl. Acad. Sci. USA, 1977; 74: 5463-5467), and relies on the use of dideoxy derivatives of the four nucleotides which are incorporated into the nascent polynucleotide chain in a polymerase reaction. Upon incorporation, the dideoxy derivatives terminate the polymerase reaction and the products are then separated by get electrophoresis and analysed to reveal the position at which the particular dideoxy derivative was incorporated into the chain.

Although this method is widely used and produces reliable results, it is recognised that it is slow, labour-intensive and expensive.

U.S. Pat. No. 5,302,509 discloses a method to sequence a polynucleotide immobilised on a solid support. The method relies on the incorporation of 3-blocked bases A, G, C and T having a different fluorescent label to the immobilised polynucleotide, in the presence of DNA polymerase. The polymerase incorporates a base complementary to the target polynucleotide, but is prevented from further addition by the 3′-blocking group. The label of the incorporated base can then be determined and the blocking group removed by chemical cleavage to allow further polymerisation to occur. However, the need to remove the blocking groups in this manner is time-consuming and must be performed with high efficiency.

WO-A-00/39333 describes a method for sequencing polynucleotides by converting the sequence of a target polynucleotide into a second polynucleotide having a defined sequence and positional information contained therein. The sequence information of the target is said to be “magnified” in the second polynucleotide, allowing greater ease of distinguishing between the individual bases on the target molecule. This is achieved using “magnifying tags”, which are predetermined units of nucleic acid sequence. Each of the bases adenine, cytosine, guanine and thymine on the target molecule is represented by an individual magnifying tag, converting the original target sequence into a magnified sequence. Conventional techniques may then be used to determine the order of the magnifying tags, and thereby determine the specific sequence on the target polynucleotide.

In a preferred sequencing method, each magnifying tag comprises a label, e.g. a fluorescent label, which may then be identified and used to characterise the magnifying tag.

WO-A-04/094664 describes an adaptation of the conversion method disclosed in WO-A-00/39333. In both methods, it is preferred that each magnifying tag comprises two units of distinct sequence which can be used as a binary system, with one unit representing “0” and the other representing “1”. Each base on the target is characterised by a combination of the two units, for example adenine may be represented by “0”+“0”, cytosine by “0”+“1”, guanine by “1”+“0” and thymine by “1”+“1”.

As with all sequencing procedures, maintaining high accuracy is essential to the success of the sequencing reaction. There is therefore a long felt need to obtain maximum accuracy from any sequencing reaction.

### SUMMARY OF THE INVENTION

The present invention provides a method of increasing the accuracy of sequencing reactions, in particular those involving the use of binary signals, for example as described in WO-A-00/39333 and WO-A-04/094664 (both of which are incorporated herein by reference), or those involving base to base signals, eg. ligation proximity assay. The invention is based on the realisation that when a sequencing reaction involves the conversion of a target molecule, eg. a polynucleotide, into a polynucleotide comprising distinct units of sequence information, the accuracy of the sequence data obtained can be improved by incorporating into the polynucleotide defined sequences that act as internal controls which can be determined to ensure the detection of sequencing errors. These control sequences do not directly represent the sequence of the target polynucleotide.

According to a first aspect of the invention, a method of identifying at least one characteristic of a target molecule, comprises the steps of:

(i) converting the at least one characteristic into a signal polynucleotide sequence; and

(ii) identifying the signal polynucleotide sequence, thereby identifying the at least one characteristic of the target molecule wherein each signal polynucleotide sequence comprises at least one control sequence that defines a characteristic of the signal polynucleotide sequence, and wherein identification of the control sequence confirms whether the signal polynucleotide sequence has been identified correctly, and, optionally, if the identification is not correct, provides the necessary information to determine what the correct signal polynucleotide sequence should be.

According to a second aspect of the present invention, a method of sequencing a target polynucleotide comprises the steps of:

(i) converting at least one base on the target polynucleotide into a signal sequence; and

(ii) identifying the signal sequence, thereby identifying the sequence of the target polynucleotide wherein each signal sequence comprises at least one control sequence that defines a characteristic of the signal sequence, and wherein identification of the control sequence confirms whether the signal sequence has been identified correctly, and optionally, if the identification is not correct, provides the necessary information to determine what the correct signal sequence should be.

### DESCRIPTION OF THE DRAWINGS

The invention is described with reference to the accompanying figures, wherein;

FIG. 1A illustrates a binary signal sequence which contains information on three bases in the target polynucleotide and two control bits; and

FIG. 1B illustrates a method of using control sequences to define the bit-content of the binary signal sequence to which it is attached, wherein bit-triplets containing no or two “0” bits are designated with control bit “0” and bit-triplets containing no or two “1” bits are designated with control bit “1”.

### DESCRIPTION OF THE INVENTION

The present invention is based on the realisation that a target molecule can be converted into a defined polynucleotide sequence and that the accuracy of the eventual read-out step can be assessed by incorporating a control sequence into the formed polynucleotide sequence, and detecting the presence or absence of the control sequence.

The method of the present invention is particularly suitable for improving the accuracy of sequencing reactions in which a target polynucleotide is converted into a second polynucleotide of defined sequence, referred to herein as a “signal sequence”. The method is based upon the realisation that adding control sequences into the signal sequence provides an internal check on the sequence data obtained and allows the identification of potential errors in the read-out step.

With reference to target molecules, it is the characteristics of the target molecules which can be represented by (converted into) the signal polynucleotide sequence. For example, if the target molecule is a protein, each amino acid monomer may be represented by a specific sequence on the signal polynucleotide sequence. In the preferred embodiment, the target molecule is a polynucleotide, and conversion is carried out by amplification of the polynucleotide sequence. The invention is further described with a polynucleotide as the target molecule.

The term “polynucleotide” is well known in the art and is used to refer to a series of linked nucleic acid molecules, e.g. DNA or RNA. Nucleic acid mimics, e.g. PNA, LNA (locked nucleic acid) and 2′-O-methRNA are also within the scope of the invention.

The reference herein to the bases A, T(U), G and C, relate to the nucleotide bases adenine, thymine (uracil), guanine and cytosine, as will be appreciated in the art. Uracil replaces thymine when the polynucleotide is RNA, or it can be introduced into DNA using dUTP, again as well understood in the art.

A “signal sequence” is a single stranded or double stranded polynucleotide that comprises distinct “units” of nucleic acid sequence. Each of the bases A, T(U), G and C on the target is represented by a distinct and predefined unit, or unique combination of units in the signal sequence. Each unit will preferably comprise 2 or more nucleotide bases, preferably from 2 to 50 bases, more preferably 2 to 20 bases and most preferably 4 to 10 bases, e.g. 6 bases. There are at least two different bases contained in each unit. The design of the units is such that it will be possible to distinguish the different units during a “read-out” step, e.g. involving the incorporation of detectably labelled nucleotides in a polymerisation reaction, or on hybridisation of complementary oligonucleotides. Sequencing methods in which the target is converted into a second polynucleotide “signal sequence” are well known in the art, for example as described in WO-A-00/39333 and WO-A-04/094664.

In a preferred embodiment of these sequencing techniques, each of the bases in the target polynucleotide is represented by two units of distinct sequence in the signal sequence. According to this embodiment, two units can be used as a binary system, with one unit representing “0” and the other representing “1”; each base in the target is thereby represented by a 2-bit binary code. Each “0” or “1” is referred to herein as a “sequence bit”. Each base on the target is characterised by a combination of the two bits. For example, adenine may be represented by “0”+“0”, cytosine by “0”+“1”, guanine by “1”+“0” and thymine by “1”+“1”. It is necessary to distinguish between the units, and so a “stop” signal can be incorporated into each unit. It is also preferable to use different units representing “1” and “0”, depending on whether the base on the target (template) polynucleotide is in an odd or even numbered position.

This is demonstrated as follows:

Odd numbered template sequence:

“0”: TTTTTTA(CCC)
“1”: TTTTTTG(CCC)

Even numbered template sequence:

“0”: CCCCCCA(TTT)
“1”: CCCCCCG(TTT)

In this example, the underlined base is the target for labelled nucleotides in a polymerase reaction, the bases in parentheses are used as a stop signal, and the remaining bases are to provide separation between the labels.

Suitable signal sequences are also described in WO-A-00/39333.

This binary method therefore involves the combination of “bits” of sequence to form a signal sequence. The method of the present invention incorporates “control bits” into the signal sequence. As used herein, the term “control bit” refers to a pre-defined unit of sequence intended to define the sequence of bits in the signal sequence to which it is adjacent; each control bit provides a summary of the sequence to which it is adjacent. During the read-out step, the information contained in the control bit is used to verify that the information read from the adjacent sequence is correct. The terms “control bit” and “control sequence” are used interchangeably.

In its simplest form, each bit in the signal sequence can be immediately followed (or preceded) by a second identical bit which acts as the control bit. In this embodiment, each sequence bit in the signal sequence is repeated by a control bit, providing an internal control and check on the eventual sequencing of the signal sequence.

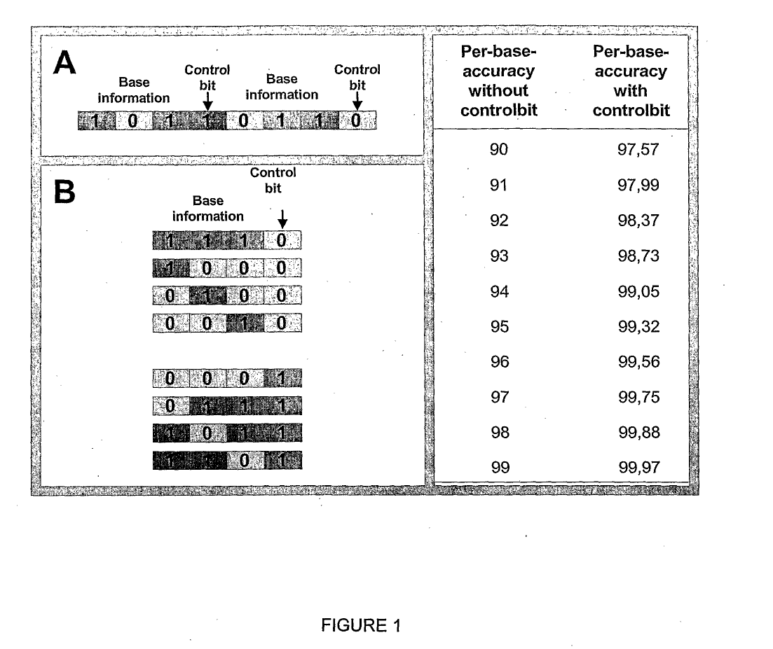

In a preferred embodiment, each control bit defines a plurality of sequence bits. Preferably, each control bit defines between 2 and 10 sequence bits, more preferably between 2 and 5 bits. Most preferably each control bit defines 3 sequence bits, as shown in FIG. 1A. When each control bit defines 3 sequence bits, the control bits can define the sequence bits as illustrated in FIG. 1B. If the triplet of sequence bits contains no, or 2, “0” bits, a “0” control bit is associated with the triplet. If the bit triplet contains 0 or 2 “1” bits, the control bit “1” is used. In this system, a single bit change in a bit triplet will always result in a change of the control bit and a misinterpretation of a bit during the read-out step, i.e. mistaking a “0” for a “1”, will be detected using the control bit (unless two bits are misinterpreted at the same time in the same triplet). If, using the preferred control system illustrated in FIG. 1B, the control bit is a “1”, this indicates that the previous triplet must contain two “1” bits and a single “0”. If one of the bases has been misread, for example a “1” has been read as a “0”, the control bit will highlight this error. The control bit therefore defines the number of each type of bit, or the “bit-content”, of each triplet of sequence bits with which it is associated. This system provides an internal control for the read-out phase.

This preferred system utilises the “parity bit” concept from the field of computer programming and applies it in the fields of molecular biology and biochemistry. In this preferred embodiment, the control bit functions as a parity bit by defining the bit-content of each triplet (or the other number) of sequence bits with which it is associated. “Odd” or “even” parity may be used, i.e. the parity (control) bit will define whether there is an odd or even number of the specified sequence bit (“0” or “1”) in the region of signal polynucleotide associated with the parity (control) bit.

The increase in accuracy gained by using one control bit for every 3 sequence bits is indicated in the table below.

| Per-base accuracy | Per-base accuracy |
|---|---|
| without control bit | with control bit |
| 90 | 97.57 |
| 91 | 97.99 |
| 92 | 98.37 |
| 93 | 98.73 |
| 94 | 99.05 |
| 95 | 99.32 |
| 96 | 99.56 |
| 97 | 99.75 |
| 98 | 99.88 |
| 99 | 99.97 |

In a preferred embodiment of the present invention, each signal sequence contains the binary information which codes for three bases in the target polynucleotide, i.e. 6 bits of information. After every third bit, a control bit is incorporated into the signal sequence, which defines the previous three bits in the sequence, as shown in FIGS. 1A and 1B. Each signal sequence therefore contains eight bits of information, six of which represent the bases in the target polynucleotide and two of which are control bits. In each cycle of “conversion” of the target polynucleotide into the signal sequence, information on 3 bases in the target is represented in the signal sequence. To sequence greater than three bases using this preferred embodiment, further cycles of signal sequence addition can be used to form a single chain comprising a defined series of signal sequences, as described in WO-A-00/39333 and WO-A-04/094664.

In an embodiment, the control bit may be of a defined sequence characteristic for a specific polynucleotide signal sequence (or portion of the sequence). If there is an error in the signal sequence, for example if an incorrect number of bases are sequenced in the read-out step, the control bit can be identified and its identity allows the identification of what the correct signal sequence (or portion of the signal sequence) should be. In this way, the control bit acts as an error correction sequence, in a similar way to error correction codes used in computer designs (for example, Hamming codes). The control bit should therefore be of a sufficient length to enable specific characterisation of the signal sequence (or portion thereof) to occur. For example, if a portion of the signal sequence corresponds to the specific nucleotide base A, the control bit should enable characterisation of the portion of the signal sequence to determine that it corresponds to A, in the event that the signal sequence is sequenced incorrectly or formed incorrectly from the original target molecule.

In addition to the control bit present in each signal sequence, the method of the invention can be carried out with the insertion of additional control bits at defined regions or intervals during the construction of the signal sequence. Having an additional control bit at regular intervals enables the user to confirm that the polynucleotide signal sequence is present in the correct format (sequence) and therefore that the conversion and/or read-out step has taken place correctly. For example, if the target molecule is a polynucleotide, and conversion takes place to sequence the target, the presence of additional control bits, expected at intervals corresponding to every 10 bases (on the target), will increase the possibility that any frame-shift is detected. If, for example, the sequencing experiment results in a frame-shift caused during the sequencing of the signal sequence, the additional control bit will not be identified after a sequence corresponding to 10 bases as expected; this indicates that there has been an error somewhere in the sequence after the last additional control bit was detected. These additional control bits may be inserted after any defined number of bases (or other characteristics) of the target. For example, they may be inserted at conversion of every 1 to 10 bases. For example, the bases A, C, G and T are represented by a binary sequence as shown below, and a control bit sequence which separates each ‘converted’ base.

| | | |
|---|---|---|
| A = | 00 | 01 |
| C = | 01 | 01 |
| G = | 10 | 01 |
| T = | 11 | 01 |

The sequence 01 is the control bit and this should be identified on sequencing the code for each base. If 01 is not identified on sequencing a base, it indicates that the read-out step has missed a sequence and so a repeated sequencing/read-out step is performed.

In a further separate embodiment, a control bit may be used to ensure that the read-out step is performed accurately when sequencing bases characterised by a series of either “0” or “1”. It may often be difficult for a read-out platform to discriminate between a series of “0” or “1” and so, rather than determine, say, four consecutive “0”, the read-out determines only three. It is therefore preferred to ensure that separation of consecutive “0” (or “1”) occurs. This can be achieved by introducing redundant control bit sequences within each sequence corresponding to a base, to ensure that only a limited number of “0” are ever consecutive. The redundant control bit is removed (usually by computer algorithm) on sequencing to identify the correct sequence.

For example, taking the binary code for A, G, C and T as indicated above, redundant control bits can be introduced as follows:

| | |
|---|---|
| A = | 01001 |
| C = | 01101 |
| G = | 11001 |
| T = | 10101 |

The underlined sequence at position 2 is the control bit. This ensures that the signal sequence does not contain either a series of 3 or more consecutive ‘0’ or 3 or more consecutive ‘1’. The read-out step can then be performed and (knowing that the redundant control bit is at position 2) the redundant control bit can be removed. The redundant control bit can be inserted at the correct position by use of the correct linker molecules, as disclosed in WO-A-04/094664.

Once a signal sequence containing at least one control sequence has been produced, it is necessary to perform, a “read-out step” to obtain the sequence information encoded within.

The read-out step may be performed using any suitable technique, for example as described in WO-A-00/39333 and WO-A-04/094663 and summarised herein. A preferred detection technique is as discussed above, using the polymerase reaction to incorporate bases complementary to those on the signal sequence, using either selected, detectably-labelled nucleotides or nucleotides that incorporate a group for subsequent indirect labelling, and monitoring any incorporation event.

To carry out the polymerase reaction-based read-out step it will usually be necessary to first anneal a primer sequence to the signal sequence polynucleotide, the primer sequence being recognised by the polymerase enzyme and acting as an initiation site for the subsequent extension of the complementary strand. The primer sequence may be added as a separate component with respect to the polynucleotide, which comprises a complementary sequence that allows the primer to anneal. The polymerase reaction is preferably carried out under conditions that permit the controlled incorporation of complementary nucleotides one unit at a time. This enables each magnified signal sequence unit to be categorised by the detection of an incorporated label. As each unit preferably comprises a “stop” sequence, it is possible to control incorporation by supplying only those nucleotides required for incorporation onto the first unit, as described above. As each unit is recognised by a specific label, it is possible to distinguish between two different units (0 and 1) within each cycle. This enables detection of any incorporated label, and allows the identification of the unit.

The read-out method may be carried out as follows:
(i) contacting the signal sequence comprising the defined units with at least one of the nucleotides dATP, dTTP, dGTP and dCTP, under conditions that permit the polymerisation reaction to proceed, wherein the at least one nucleotide comprises a detectable label specific for that nucleotide;
(ii) removing any non-incorporated nucleotides and detecting any incorporation events;
(iii) removing the labels from incorporated nucleotide; and
(iv) repeating steps ii) to iv), to thereby identify the different units, and thereby the sequence of the target polynucleotide.

The number of different nucleotides required in step (i) of each cycle will be dependent on the design of the signal sequence units. If each unit comprises only one base type, then only one nucleotide (detectably labelled) is required. However, if two bases are utilised (one as a target for the detectably labelled nucleotide and one to provide a gap between different target bases) then two nucleotides will be required (one to bind to the target base and one to “fill in” the bases between the target bases).

The use of a base as a stop signal allows the detection steps to be performed without the requirement for blocked nucleotides to prevent uncontrolled incorporation during the polymerase reaction. The stop signal is effective as the complement for the “stop” base is absent from the polymerase mix. Therefore, each unit can be characterised before a “fill-in” step is performed, using the missing nucleotide, to incorporate a complement to the stop base, which allows the next unit to be characterised. This is carried out after the detection step. The “stop” base of one unit will not be of the same type as the first base of the subsequent unit. This ensures that the “fill-in” procedure does not progress to the next unit. Non-incorporated nucleotides used in the “fill-in” procedure can then be removed, and the next unit can then be characterised.

The choice of polymerase and detectable label will be apparent to the skilled person. The following is used as a guide only:

1. Klenow and Klenow (exo−) can efficiently incorporate Tetramethylrhodamine-4-dUTP and Rhodamin-110-dCTP (Amersham Pharmacia Biotech) (Brakmann and Nieckchen, 2001, Brakmann and Lobermann, 2000).
2. Vent, Taq and Tgo DNA polymerase can efficiently incorporate dioxigenin and fluorophores like AMCA, Tetramethylrhodamin, fluorescein and Cy5 without spacing at least up to a few positions (Augustin et al., (provide reference?) 2001).
3. T4 DNA polymerase is efficient in filling-in fluorophore labelled nucleotides.

The preferred polymerases are Klenow Large fragment (exo−) and T4 DNA polymerase.

Other conditions necessary for carrying out the polymerase reaction, including temperature, pH, buffer compositions etc., will be apparent to those skilled in the art. The polymerisation step is likely to proceed for a time sufficient to allow incorporation of bases to the first unit. Non-incorporated nucleotides are then removed, for example, by subjecting the array to a washing step, and detection of the incorporated labels may then be carried out.

An alternative read-out strategy is to use short detectably labelled oligonucleotides to hybridise to the units on the magnified readable signal sequence and/or positional tag, and to detect any hybridisation event. The short oligonucleotides have a sequence complementary to specific units of the readable signal sequence. For example, if a binary system is used and each monomer in the sample fragment is defined by a different combination of signal sequence units (one representing “0” and one representing “1”) the invention will require an oligonucleotide specific for the “1” unit. In this embodiment, selective hybridisation of oligonucleotides can be achieved by designing each unit to be of a different polynucleotide sequence with respect to other units. This ensures that a hybridisation event will only occur if the specific unit is present, and the detection of hybridisation events identifies the characteristics on the sample fragment.

In a preferred embodiment, the label is a fluorescent moiety. Many examples of fluorophores that may be used are known in the prior art, as indicated above. The attachment of a suitable fluorophore to a nucleotide can be carried out by conventional means. Suitably labelled nucleotides are also available from commercial sources. The label is attached in a way that permits removal, after the detection step. This may be carried out by any conventional method, including:

I. Attacking the signal itself:

### 4) Bleaching

1) Photobleaching
2) Chemical bleaching
   2) Quenching of fluorescence
i) By antibodies raised against the fluor (e.g. anti-fluorescein, anti-Oregon green)
ii) By FRET (the incorporation of a quencher next to a signal can be used to quench the signal, e.g. Taqman strategy)
   3) Cleavage of signal
i) Chemical cleavage (e.g. reduction of a disulfide bridge between the base and the signal)
ii) Photocleavage (e.g. introduction of a nitrobenzyl ortert-butylketoh group)
iii) Enzymatic (e.g. a-chymotryspin digestion of peptide linker)
   II. The signal bearing nucleotide:
   1) Exonucleolytic removal
i) 3′-5′ Exonucleolytic degradation of filled-in nucleotides (e.g. exonuclease III or by activating the 3′-5′ exonucleolytic activity of DNA polymerase when there is an absence of certain nucleotides)
   2) Restriction enzyme digestion
ii) Digestion of double-stranded DNA bearing the signal (e.g. ApaI, DraI, SmaI sites which can be incorporated at the stop signals).

An alternative to the use of labels that permit removal, is to use inactivated labels that are reactivated during a biochemical process.

The preferred method is by photo or chemical cleavage.

When the label is a fluorophore, the fluorescent signal generated on incorporation may be measured by optical means, e.g. by a confocal microscope. Alternatively, a sensitive 2-D detector, such as a charge-coupled detector (CCD), can be used to visualise the individual signals generated.

The general set-up for optical detection is as follows:

| | |
|---|---|
| Microscope: | Epi-fluorescence |
| Objective: | Oil emersion (100X, 1.3 NA) |
| Light source: | Lasers or lamp |
| Filters: | Bandpass |
| Mirrors: | Dichroic mirror and dichroic wedge |
| Detectors: | Photomultiplier tubes (PMT) or CCD camera |

Variants may also be used, including:

A. Total Internal Reflection Fluorescence Microscopy (TIRFM)

| | |
|---|---|
| Light source: | One or more lasers |
| Background | No pinhole required |
| control: | |
| Detection: | CCD camera (video and digital imaging systems) |

B. Confocal Laser Scanning Microscopy (CLSM)

| | |
|---|---|
| Light source: | One or more lasers |
| Background | One or several pinhole apertures |
| reduction: | |
| Detection: | a) A single pinhole: Photomultiplier tube (PMT) |
| | detectors for different fluorescent wavelengths [The |
| | final image is built up point by point and overtime by |
| | a computer]. b) Several thousands pinholes (spinning |
| | Nipkow disk): CCD camera detection of image [The |
| | final image can be directly recorded by the camera] |

C. Two-Photon (TPLSM) and Multiphoton Laser Scanning Microscopy

| | |
|---|---|
| Light source: | One or more lasers |
| Background | No pinhole required |
| control: | |
| Detection: | CCD camera (video and digital imaging systems) |

The preferred methods are TIRFM and confocal microscopy.

The read-out platform may also be based on nanopores as disclosed in WO00/39333, the content of which is incorporated herein by reference.

It will be appreciated that although specific examples of techniques suitable for read-out of the signal sequence are given herein, the signal sequences may be read using any suitable read-out platform.

I claim:

1. A method of identifying at least one characteristic of a target molecule, comprising:
(i) converting the at least one characteristic into a signal polynucleotide; and
(ii) identifying the signal polynucleotide sequence, thereby identifying the at least one characteristic of the target molecules wherein each signal polynucleotide comprises at least one control sequence that defines a characteristic of the signal polynucleotide, and wherein identification oil the control sequence confirms whether the signal polynucleotide sequence has been identified correctly, and, optionally, if the identification is not correct, provides the necessary information to determine what the correct signal polynucleotide sequence should be.

2. The method according to claim 1, wherein the target molecule is a polymer.

3. The method according to claim 2, wherein the characteristic to be identified is at least one monomer.

4. The method according to claim 3, wherein the at least one monomer is a nucleotide.

5. The method according to claim 1, wherein each characteristic of the target polymer is represented by at least one distinct unit of sequence in the signal polynucleotide.

6. The method according to claim 5, wherein characteristic of the target polymer is represented by a specific combination of two or more distinct polynucleotide sequence units in the signal polynucleotide.

7. The method according to claim 6, wherein each characteristic of the target polymer is represented by a specific combination of two or more polynucleotide sequence units designated “0” and “1” in the signal polynucleotide, thereby creating a binary signal polynucleotide.

8. The method according to claim 2, wherein three monomers on the target polymer are converted into a signal polynucleotide in (i).

9. The method according to claim 1, wherein control sequences are incorporated into the signal polynucleotide at predetermined intervals.

10. The method according to claim 5, wherein a control sequence is incorporated into the signal polynucleotide after every third unit of sequence.

11. The method according to claim 6, wherein the control sequence defines the combination of units with which it is associated.

12. The method according to claim 7, wherein the control sequence is a “0” or “1” unit that defines the number of “0” or “1” units in the region of the signal polynucleotide with which it is associated.

13. The method according to claim 7, wherein the control sequence is present in the signal sequence in a defined position such that there are no more than three sequence units of the same type representing the characteristics of the target.

14. The method according to claim 1, wherein (i) and (ii) are repeated to form a molecule having a series of polynucleotide signal sequences representing the characteristics of the target molecule.

15. The method according to claim 13, wherein additional control sequences are incorporated at defined intervals into the molecule formed, so that identification of the additional control sequences reveals whether the correct number of signal sequences have been incorporated.

16. A method of sequencing a target polynucleotide, comprising:
(i) converting at least one base on the target polynucleotide into a signal sequence; and
(ii) identifying the signal sequence, thereby identifying the sequence of the target polynucleotide wherein each signal sequence comprises at least one control sequence that defines a characteristic of the signal sequence, and wherein identification of the control sequence confirms whether the signal sequence has been identified correctly.

17. The method according to claim 16, wherein if the identification is not correct, the control sequence provides the necessary information to determine what the correct signal sequence should be.
